(19) United States
(12) Patent Application Publication
(10) Pub. No.: US 2011/0082479 A1
(43) Pub. Date: Apr. 7, 2011

(54) Apparatus, method and system for the deployment of surgical mesh

(76) Inventor: Jack Friedlander, Rocklin, CA (US)

(21) Appl. No.: 12/587,458

(22) Filed: Oct. 7, 2009

(51) Int. Cl. A61B 17/00

(52) U.S. Cl. 606/151

(57) ABSTRACT

An apparatus, method and system for the deployment of surgical mesh material, which are particularly suited for the use in the laparoscopic surgical repair of hernias. An inner actuator rod slides within a main shaft. Mesh deployment arms are connected to an end of the shaft, and a surgical mesh is mounted to and rolled around the deployment arms. An outer housing slides over the main shaft, the deployment arms, and conformed mesh. By sliding the main shaft in a first direction, the mesh is exposed and the actuator rod is retracted, which actively flexes the deployment arms, thereby unfurling the mesh. By sliding the main shaft in an opposite direction, the to tension on the deployment arms is relaxed, and the deployment arms disengage from the actuator rod allowing the deployment apparatus to be withdrawn from the mesh.

### BACKGROUND OF THE INVENTION

1. Field of the Invention

The present invention relates generally to surgical devices, and more particularly to an apparatus, method and system for the deployment of a surgical mesh inside of a patient.

2. Description of the Related Art

Hernias are defects in the abdominal wall connective tissue (fascia) through which intra-abdominal contents can protrude. Surgical repair is necessary in order to prevent complications of incarceration (contents become trapped in the defect) and strangulation (blood supply to the contents becomes compromised) which can lead to significant morbidity and potential mortality of a patient. Inguinal hernias are defects in the lower abdominal wall and are the most common types of hernias, and thus repair of inguinal hernias is one of the most commonly performed general surgical procedures. Another type of hernia is termed a ventral or incisional hernia, which forms in the anterior abdominal wall and frequently occurs at the site of a previous operative incision, though they may also occur without prior surgery.

Laparoscopic surgery is a well-known, widely utilized surgical technique that advantageously reduces patient recovery time due to its minimal tissue damage. Laparoscopic surgery is performed by inflating the abdominal cavity with carbon dioxide gas followed by insertion of a number of thin cannulas through the abdominal wall. A video scope is placed through one of the cannulas, and long thin operating instruments are placed through other cannulas. The cannulas commonly used in laparoscopic surgery have inner diameters of 5 millimeters (mm), 10 mm, 12 mm, and less commonly 15 mm.

Hernias of the abdominal wall can be repaired using laparoscopic techniques by the placement of a surgical reinforcement prosthesis (i.e. a surgical mesh material) inside the abdominal cavity or in the floor of the inguinal canal, and then against the hernia defect. This procedure repairs the hernia defect and imparts the aforementioned advantages of laparoscopic surgery to the repair of abdominal wall and inguinal hernias.

Laparoscopic ventral hernia repair is performed in several steps:
1. Preparation of the mesh on the outside of the patient, which must include rolling, folding or otherwise conforming the mesh such that it may be passed through a surgical cannula into the patient, and usually includes placement of orientation or fixation sutures into the mesh for use in later portions of the procedure.
2. Passage of the mesh into the patient through a surgical cannula of limited internal diameter without damaging the mesh or causing harm to the patient.
3. Unfurling of the mesh in the proper orientation inside the patient. This must be performed such that the proper surface of the mesh is facing up toward the abdominal wall, and such that the mesh is properly oriented to the size, shape and location of the hernia defect in the abdominal wall. This is normally accomplished by grasping and manipulating the mesh with laparoscopic surgical graspers.
4. Once oriented, the mesh must be elevated up to the abdominal wall where the hernia defect resides. This may be performed using surgical graspers and four point traction, or by the use of the previously placed orientation or fixation sutures. These sutures may be grasped by a long thin suture grasping device, which is placed through the abdominal wall through separate incisions, and draws the fixation sutures up through the abdominal wall at four separate points, lifting the mesh up to the abdominal wall. The fixation sutures may then be tied in place or otherwise affixed in order to approximate the mesh, in proper orientation, and under proper tension, to the abdominal wall.
5. Securing the mesh to the abdominal wall is normally accomplished by the application of a tacking device or other affixation means.

Numerous difficulties are encountered in laparoscopic hernia repair in the first four phases of the procedure described above. Preparation of the mesh on the outside of the patient may include placement of orienting or fixation sutures onto the edges of, or in the center of the mesh, adding to the operative time, and must include rolling, folding or otherwise conforming the mesh to fit within the inner diameter of the surgical cannula through which it must be passed.

The bulk of the mesh/prosthesis material limits the size of the same which can be passed through a given sized cannula. The mesh must be conformed, by rolling, folding or other manipulations such that it can be passed through the cannula to the inside of the patient without causing damage to the mesh or injury to the patient. In some cases the mesh is so large that it must be passed through an enlarged skin incision directly, without passage through a cannula, risking contamination of the mesh with infectious agents which may be present on the skin, risking organ and or tissue damage, and significantly increasing the operating time.

The lack of rigidity of mesh/prosthetic materials makes passage of a conformed sheet of mesh/prosthesis through a surgical cannula difficult without some additional rigidity. In some surgical practices, the mesh is rolled around a laparoscopic surgical instrument in order to impart this rigidity. A problem with this approach is that the diameter of the surgical instrument adds significantly to the final outer diameter of the rolled or conformed mesh, again limiting the size of mesh prosthesis which can be placed through the fixed inner diameter of the cannula.

Another problem relates to unfurling and orienting the mesh prosthesis in the patient, which is typically performed by the use of laparoscopic grasping instruments and is aided by placement of orienting sutures. Proper orientation of the mesh/prosthesis must include unfurling of the mesh with the correct side of the mesh facing upwards towards the abdominal wall, and must also include orienting the mesh to the size, shape and position of the hernia defect, a process which is both difficult and time consuming.

Elevation of the mesh prosthesis up to the anterior portion of the abdominal wall where the hernia defect resides is usually performed by passing a long thin suture grasping instrument through the abdominal wall, grasping each of the four fixation sutures, and pulling them up through the abdominal wall. Grasping and manipulating the sutures requires significant laparoscopic surgical skills not possessed by a majority of surgical practitioners. Also, it is often difficult to properly orient and tension the mesh using this technique. Thus, it would be desirable to have a mesh deployment system that has: 1) a small enough diameter to allow the device holding the mesh to fit through the limited diameter of a cannula; 2) an unfurling mechanism that provides ease of unfurling of the mesh, as well as proper placement and orientation of the mesh; and 3) allows the mesh to be easily disengaged from the apparatus.

### SUMMARY OF THE INVENTION

In general, the present invention is an apparatus, method and system for the deployment of surgical mesh material, which are particularly suited for the use in the laparoscopic surgical repair of hernias.

According to one embodiment of the invention, a surgical mesh deployment apparatus comprises a hollow main shaft having a slot on a proximal end, an actuator rod positioned in the main shaft, the actuator rod having an actuating pin on a proximal end engaged through the slot in the main shaft, and an actuator tip at a distal end, a mounting plug connected to an end of the main shaft, the mounting plug having to a hole for the actuator rod, two deployment arms connected to the mounting plug, wherein each deployment arm has a tip configured to engage the actuator tip, and a hollow outer housing mounted over the main shaft, the outer housing having an interior diameter larger than an exterior diameter of the main shaft and mounting plug, such that the outer housing can slide along the main shaft and over the mounting plug and deployment arms.

According to various embodiments, the deployment apparatus may further comprise a flexible tube mounted between the main shaft and the mounting plug. The actuator rod comprises a first rod including the actuating pin, a second rod including the actuator tip, and a flexible joint connecting the first and second rods. The deployment apparatus further comprises a handle attached to the main shaft at an end opposite the flexible tube, wherein the outer housing further comprises a grip on one end, the grip including a notch to engage the actuating pin. The deployment arms are connected to the mounting plug by pins, which allow the deployment arms to pivot on the mounting plug. The actuator tip may comprise two notches, wherein each deployment arm comprises a U-shaped tip for engaging a notch of the actuator tip. The actuator tip may also comprise a cap with two slots, wherein each deployment arm comprises a pointed tip a rounded tip or a square tip for engaging a slot in the actuator tip cap. The mounting plug is preferably keyed to the actuator rod to prevent rotation of the actuator rod about a long axis.

According to another embodiment of the present invention, a surgical mesh deployment system comprises a surgical mesh for insertion into a patient, a surgical mesh deployment apparatus, the apparatus comprising a hollow main shaft having a slot on a proximal end, an actuator rod positioned in the main shaft, the actuator rod having an actuating pin on a proximal end engaged through the slot in the main shaft, and an actuator tip at a distal end, a mounting plug connected to an end of the main shaft, the mounting plug having a hole for the actuator rod, two deployment arms connected to the mounting plug, wherein each deployment arm has a tip configured to engage the actuator tip, and a hollow outer housing mounted over the main shaft, the outer housing having an interior diameter larger than an exterior diameter of the main shaft and mounting plug, such that the outer housing can slide along the main shaft and over the mounting plug and deployment arms, wherein the surgical mesh is mountable on the deployment arms for deployment in a patient.

The system may further comprise a flexible tube mounted between the main shaft and the mounting plug. The actuator rod comprises a first rod including the actuating pin, a second rod including the actuator tip, and a flexible joint connecting the first and second rods. The deployment system further comprises a handle attached to the main shaft at an end opposite the flexible tube, wherein the outer housing further comprises a grip on one end, the grip including a notch to engage the actuating pin. The deployment arms are connected to the mounting plug by pins, which allow the deployment arms to pivot on the mounting plug. The surgical mesh may be formed as an oval and comprises a plurality of guide loops. The surgical mesh may further comprise a pocket to receive the actuator tip. In another embodiment, the surgical mesh comprises two parallel oval sheets attached along the edges thereof with an opening on one end.

A method of deploying a surgical mesh according to one embodiment of the present invention includes mounting a surgical mesh on two deployment arms of a deployment apparatus, engaging the two deployment arm tips with an actuator tip of an actuator rod, the deployment arms attached to a fixed mounting plug at a first end, and releasably engaged with the actuator tip of the actuator rod at a second end, sliding an outer housing over the surgical mesh and deployment arms to cover the mesh, inserting the outer housing into a patient, sliding a handle of the deployment apparatus forward to expose the surgical mesh and causing an actuating pin to engage a notch in a grip, further sliding the handle forward causing a main shaft to slide over the actuator rod, which causes the deployment arms to expand outward to unfurl the surgical mesh, pulling the handle back to release the deployment arm tips from the actuator rod tip, the actuating pin being engaged in the notch in the grip, and removing the deployment apparatus from the patient.

### BRIEF DESCRIPTION OF THE DRAWINGS

The present invention will be readily understood by the following detailed description in conjunction with the accompanying drawings, wherein like reference numerals designate like structural elements, and in which:

FIG. 1A is an exploded view of an embodiment of a mesh deployment apparatus according to the present invention;

FIG. 1B illustrates an embodiment of a surgical mesh for use with the system of the present invention;

FIG. 1C illustrates an alternate embodiment of a surgical mesh for use the system of the present invention;

FIGS. 2A-2E illustrate the operation of the deployment apparatus of FIG. 1A and the mesh of FIG. 1B;

FIGS. 3A and 3B are side and bottom views of the actuator rod assembly of FIG. 1A;

FIGS. 4A and 4B show an enlarged view of the actuator tip of the deployment apparatus of FIG. 1A;

FIG. 4C shows an enlarged view of the deployment arms engaged with the actuator rod tip;

FIGS. 5A-5D illustrate various embodiments for the actuator rod tip and deployment arm tips;

FIG. 6 shows an enlarged view of the underside of the grip; and

FIGS. 7A-7D illustrate the operation of the actuating pin and the notch in the grip.

### DETAILED DESCRIPTION OF THE INVENTION

The following description is provided to enable any person skilled in the art to make and use the invention and sets forth the best modes contemplated by the inventor for carrying out the invention. Various modifications, however, will remain readily apparent to those skilled in the art. Any and all such modifications, equivalents and alternatives are intended to fall within the spirit and scope of the present invention.

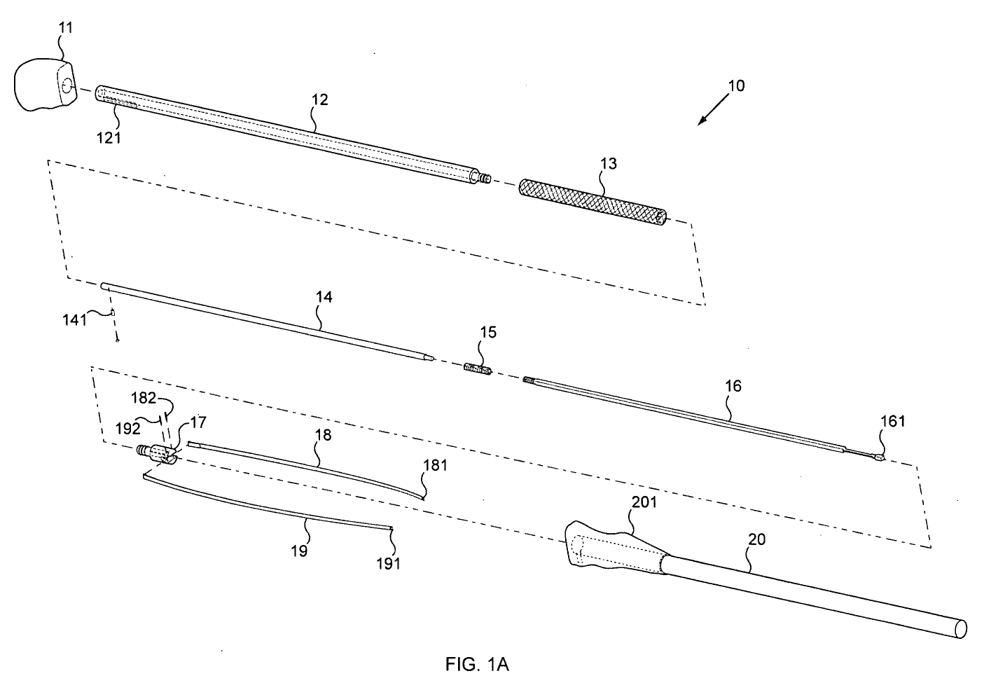

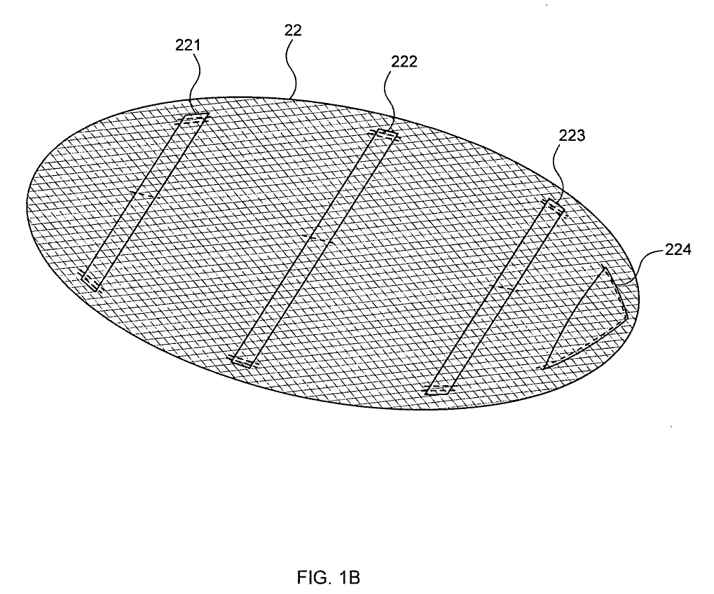

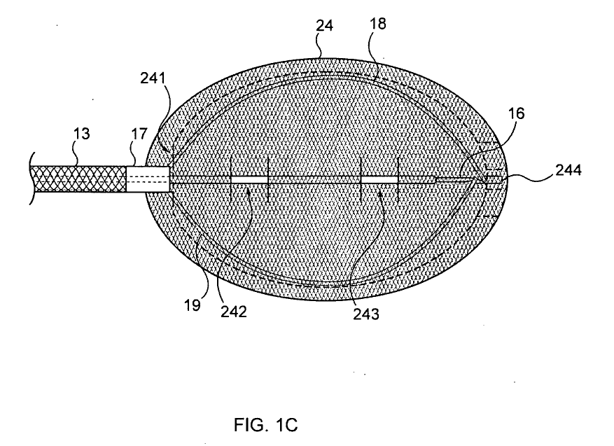

FIG. 1A is an exploded view of a mesh deployment apparatus 10 according to an embodiment of the present invention. The apparatus includes a main shaft 12 having a handle 11 affixed to one end. The main shaft 12 is preferably made from metal or biocompatible plastic. The main shaft 12 is hollow and includes a slot 121 near the handle 11, for receiving an actuating pin 141. A hollow flexible tube 13 is attached to the other end of the main shaft 12. The flexible tube 13 provides flexibility to the apparatus 10 allowing for a greater range of motion to position and manipulate the mesh.

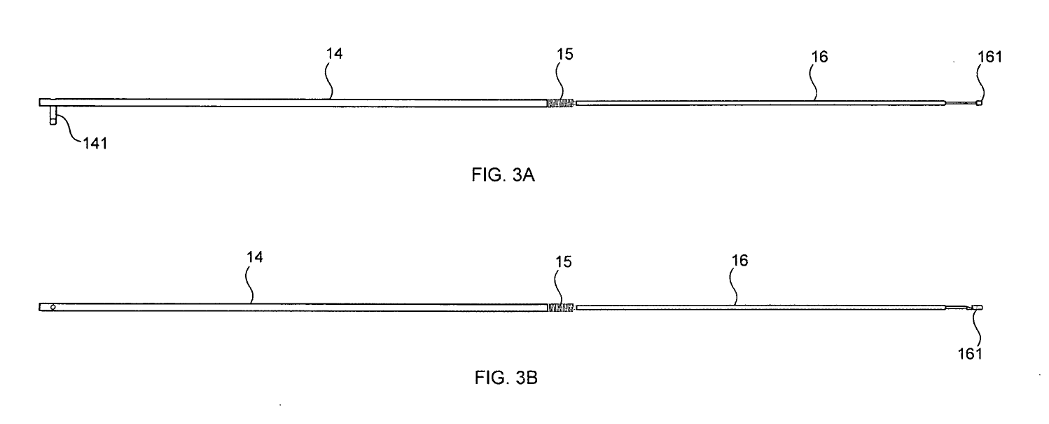

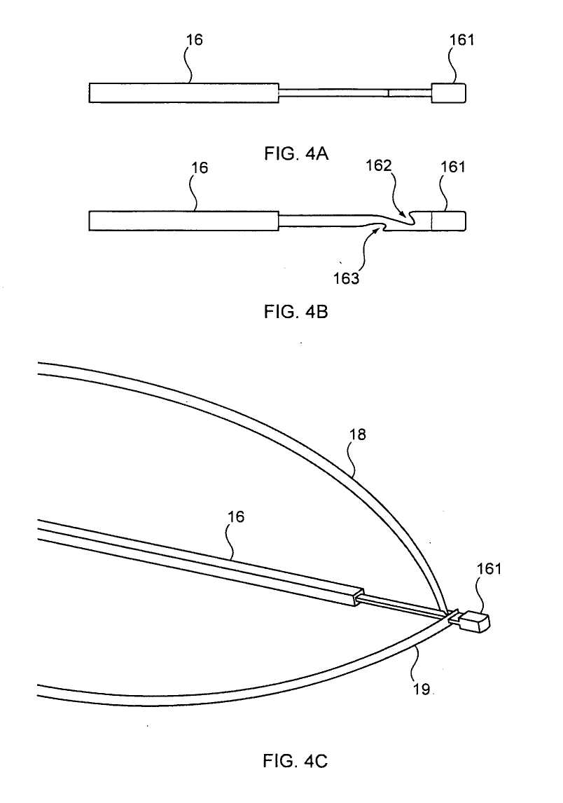

A connecting rod 14 is connected to an actuator rod 16 via a flexible joint 15. The flexible joint 15 is preferably made from a spring or other elastic material. The connecting rod 14 includes an actuating pin 141 that protrudes from slot 121 in the main shaft 12. The flexible joint 15 aligns with the hollow flexible tube 13 when the device is in the deployed position allowing the apparatus 10 to flex for proper positioning of the mesh during use. On the distal end of the actuator rod 16 is a specialized tip 161, shown in detail in FIGS. 4A-4C, and described in greater detail below. The connecting rod 14, flexible joint 15, and actuator rod 16 are positioned internally to the main shaft 12 and flexible tube 13. Thus, the respective outer diameters of the rods 14, 16 and joint 15 are smaller than the internal diameters of the hollow main shaft 12 and flexible tube 13.

A deployment arm mounting plug 17 is affixed to an end of the flexible tube 13. The mounting plug 17 includes an internal opening to allow the actuator rod 16 to slide through the mounting plug 17 and is preferably keyed to the actuator rod 16 to prevent rotation about the long axis. Two deployment arms 18, 19 are attached to the mounting plug 17. The deployment arms 18, 19 are preferably formed from spring steel, or similar elastic material which can return to its original shape after flexing. The deployment arms 18, 19 are preferably mounted symmetrically to the plug 17, in the same plane and on opposite sides of the plug 17. The deployment arms 18, 19 are further preferably attached with hinge pins 182, 192, respectively, to allow the deployment arms 18, 19 to freely pivot outward from the mounting plug 17. Each deployment arm 18, 19 includes a notched tip 181, 191, respectively, which is described in greater detail below.

Finally, a hollow outer housing 20, including a grip 201, is positioned to slidingly engage the outer surfaces of the main shaft 12 and flexible tube 13. The outer housing 20 may be manufactured from metal, or any similar rigid material. The mounting plug 17 and deployment arms 18, 19 similarly slide though the interior of the hollow outer housing 20.

Different variations of the above-described embodiment may be utilized. For example, for certain applications, it may not be necessary to have the flexible tube 13 and/or flexible joint 15 and they can be eliminated. Furthermore, different materials may be substituted to construct the various components, as is known in the art.

In a preferred embodiment, the deployment apparatus has an overall length of approximately 30 inches, and the deployment arms 18, 19 are approximately 9 inches long. A preferred surgical mesh is an oval approximately 6×9 inches. Other dimensions and shapes are within the scope of the present invention.

FIG. 1B illustrates a surgical mesh 22 according to an embodiment of the present invention, specifically configured to be inter-operable with the deployment apparatus 10 of FIG. 1A. The mesh 22 is preferably formed as an oval from standard surgical mesh material, such as polypropylene, e-PTFE or other biocompatible materials, and may be coated with any number of adhesion minimizing coatings. Guide loops 221, 222, 223 are attached to the oval mesh 22 to receive the deployment arms 18, 19. The mesh preferably contains a pouch 224 at one end for inserting the to tip of the actuator rod 161. The guide loops may be sewn or otherwise affixed to the surface of the mesh. Attachment points are preferably along the centerline of the mesh to prevent rotation of the mesh, and at the periphery to allow complete unfurling of the mesh.

Note that other suitable mesh configurations may be utilized in the present invention. For example, as shown in FIG. 1C, the mesh 24 could be formed from two oval mesh sheets attached at the outer edges to form an oval pocket, with an opening 241 on one end to receive the deployment arms 18, 19 and actuator rod 16. Center guide loop(s) 242, 243 may be attached to, or formed from the mesh 24 in the center to guide the actuator rod 16, wherein guide loops for the deployment arms 18, 19 would not be necessary in this implementation. This embodiment may also include a pocket 244 for the actuator rod tip 161.

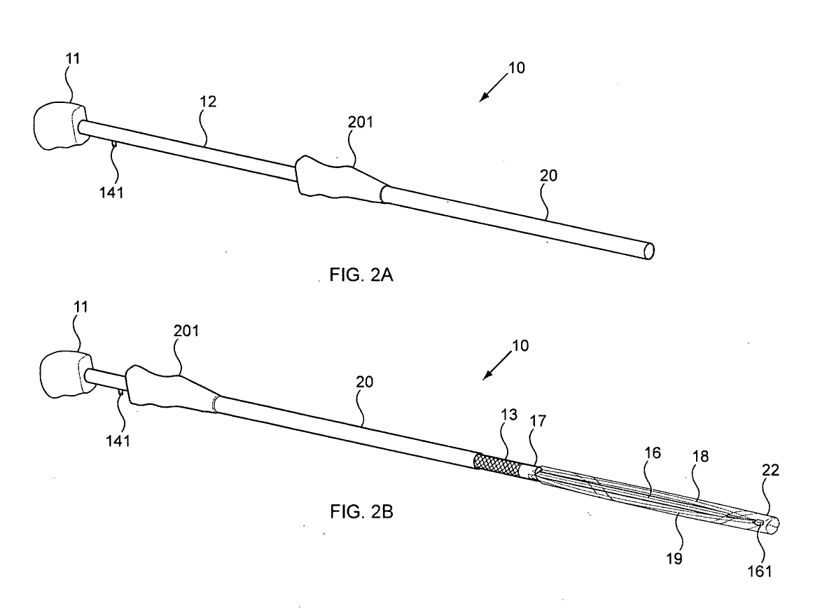

The operation of the fully assembled deployment apparatus 10 is illustrated in FIGS. 2A-2E. As shown in FIG. 2A, the outer housing 20 is slid out to cover the actuator rod 16, deployment arms 18, 19 and mesh 22. Note that in this position, the deployment apparatus 10 can easily be inserted into and positioned through a surgical cannula. Once the outer housing 20 of the deployment apparatus 10 is positioned as desired, the surgeon pushes the handle 11 inward, while holding the grip 201 on the outer housing 20. As the handle 11 is pushed inward, as illustrated in FIG. 2B, the mesh 22 is exposed. A notch 202 (described in detail below) in the end of the grip 201 engages the actuating pin 141, on the connecting rod 14. Once engaged, further inward motion of the handle 11 causes the main shaft 12 with attached flexible tubing 13 and mounting plug 17 to slide over the connecting rod 14, flexible joint 15 and actuator rod 16 while the actuating pin 141 is allowed to slide within the slot 121 in the main shaft 12. As the main shaft 12 is slid over the connecting rod 16, flexible joint 15 and actuator rod 16, the deployment arms 18, 19, whose tips 181, 191 are engaged with the specialized tip 161 of the actuator rod 16, are actively flexed and bowed outward, as shown in FIG. 2C. As the deployment arms 18, 19 bow outward, the surgical mesh 22 is unfurled until it is generally flat and parallel to the surface to which it is to be adhered. The deployment arms 18, 19 extend outward to the edge of the guide loops to completely flatten the mesh 22.

Once the mesh 22 has been unfurled, positioned as desired, and affixed to the body structure, the handle 11 is pulled outward. This releases the force on the mounting plug 17 and deployment arms 18, 19, causing the deployment arms 18, 19 to return to their neutral configuration. The actuating pin 141 having been previously engaged into the slot 202 in the grip 201 fixes the connecting rod 14, flexible joint 15, and actuator rod 16 to the grip 201, allowing the main shaft 12, flexible tubing 13, mounting plug 17 and the deployment arms 18, 19 to move outward relative to the actuator rod 16. This allows the deployment arm tips 181, 191 to fully disengage from the actuator rod tip 161, as illustrated in FIGS. 2D and 2E. Further outward motion of the handle 11 disengages the actuating pin 141 from the notch 202 and allows the actuator rod 16 and deployment arms 18, 19 to be drawn back into the housing 20 for safe removal of the apparatus from the patient.

The mesh 22 is initially mounted on the actuator rod 16 and deployment arms 18, 19 in the following manner. The actuating pin 141 is slid forward to extend the actuator rod 16 to its maximum extended position. The deployment arms 18, 19 are fed between the main mesh surface and the guide loops 221, 222, 223. Once the deployment arm tips 181, 191 are passed through the last guide loop 223, the deployment arm tips 181, 191 are engaged with the actuator rod tip 161. Next, the actuator rod tip 161 is preferably inserted into a mesh pouch 224 at an end of the mesh 22. Note that in this configuration the deployment arms 18, 19 are under some tension, but are still generally parallel to the actuator rod 16. Finally, the mesh 22 is rolled around, folded around, or otherwise configured around the deployment arms 18, 19 and actuator rod 16 (as illustrated in FIG. 2B). The outer housing 20 is slid over the assembly to fully encapsulate the mesh 22, as illustrated in FIG. 2A, and the system is now ready for deployment.

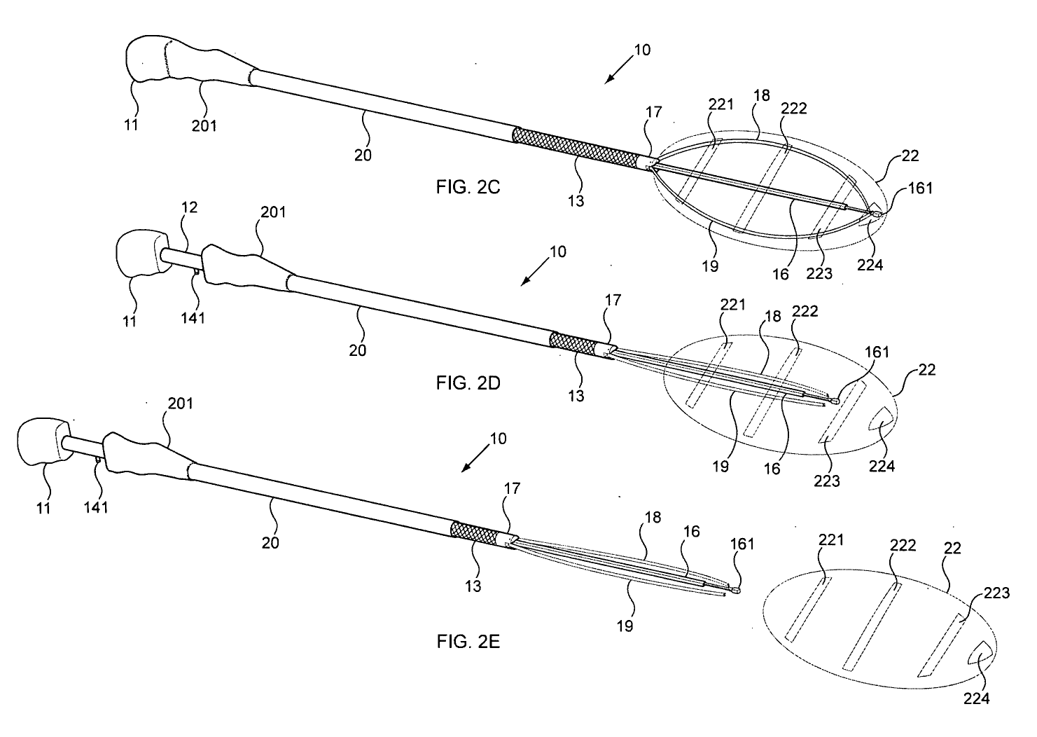

FIG. 3A illustrates the connecting rod 14, flexible joint 15, and actuator rod 16 in side profile. Not that the actuating pin 141 attaches to the connecting rod 14 to slide the entire unit through the main shaft 12, flexible tube 13 and mounting plug 17. FIG. 3B is a bottom view of FIG. 3A.

FIGS. 4A and 4B are enlarged views of the actuator rod tip 161 to show its detail. Note that the actuator rod tip 161 has notches 162, 163 on each side to receive the deployment arm tips 181, 191. Each deployment arm 181, 191 has a generally U-shaped tip to engage a notch on the actuator rod tip 161. FIG. 4C is an enlarged view of the deployment arms 18, 19 engaged with the actuator rod tip 161.

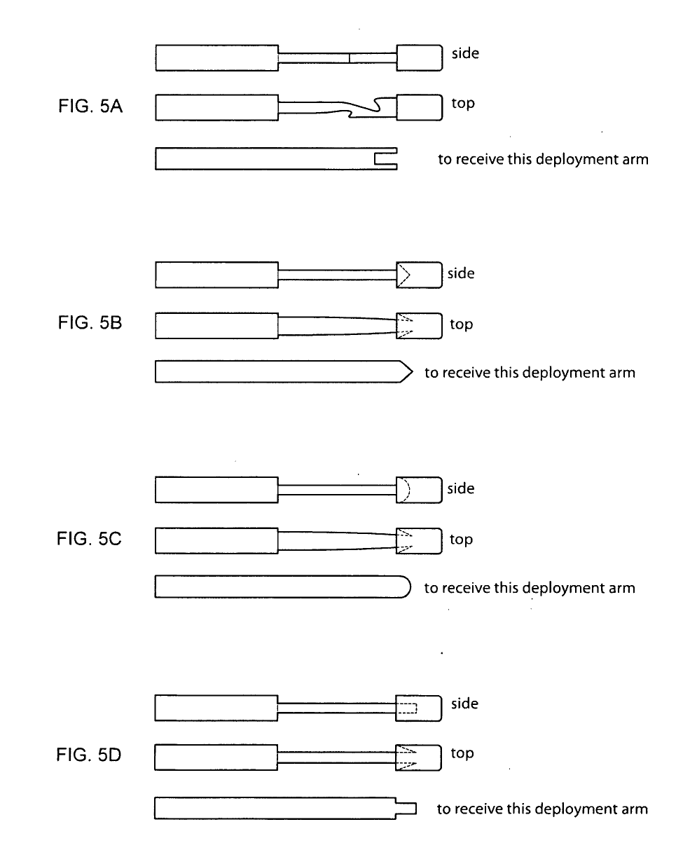

While the present invention has been illustrated with the preferred tip configuration of FIGS. 4A-4C and 5A, the actuator rod tip 161 and deployment arm tips 181, 191 may be formed in alternative configurations, as illustrated in FIGS. 5B-5D. The specific design is not critical, as long as the tips allow for easy and secure installation and deployment of the mesh 22. Thus, different configurations that allow the deployment arm tips 181, 191 to securely engage with, and fully disengage from the actuator tip 161 are within the scope of the present invention. For example, in FIGS. 5B-5D, the actuator tip 161 is formed as a cap having two slots. The slots can be configured to engage with deployment arm tips 181, 191 configured as pointed tips, rounded tips or square tips.

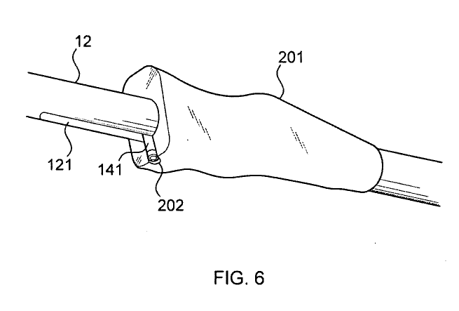

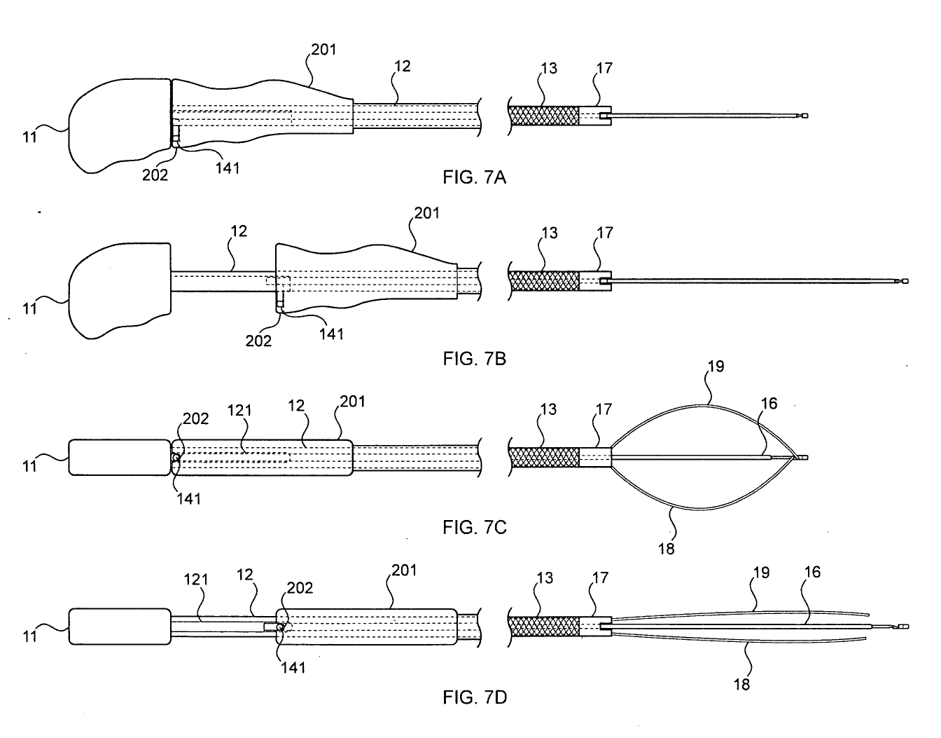

FIG. 6 illustrates the location of the notch 202 in the end of the grip 201, for engaging the actuating pin 141. FIGS. 7A-7D further illustrate in detail the operation and engagement of the notch 202 and actuating pin 141 during deployment. FIGS. 7A and 7C illustrate the deployment apparatus 10 with the actuator rod 16 fully retracted. Note that the actuating pin 141 is in the notch 202 of the grip 201. In this configuration, the actuator rod 16 is retracted which causes the deployment arms 18, 19 to expand outward. In FIGS. 7B and 7D, the handle 11 is retracted, and the actuating pin 141 is engaged with the notch 202 in the grip 201 (FIG. 7B). This causes the actuator rod 16 to extend, thereby releasing the tips 181, 191 of the deployment arms 18, 19 from the actuator rod tip 161

Those skilled in the art will appreciate that various adaptations and modifications of the just described preferred embodiments can be configured without departing from the scope and spirit of the invention. Therefore, it is to be understood that, within the scope of the appended claims, the invention may be practiced other than as specifically described herein.

What is claimed is:

1. A surgical mesh deployment apparatus comprising:
a hollow main shaft having a slot on a proximal end;
an actuator rod positioned in the main shaft, the actuator rod having an actuating pin on a proximal end engaged through the slot in the main shaft, and an actuator tip at a distal end;
a mounting plug connected to an end of the main shaft, the mounting plug having a hole for the actuator rod;
two deployment arms connected to the mounting plug, wherein each deployment arm has a tip configured to engage the actuator tip; and
to a hollow outer housing mounted over the main shaft, the outer housing having an interior diameter larger than an exterior diameter of the main shaft and mounting plug, such that the outer housing can slide along the main shaft and over the mounting plug and deployment arms.

2. The deployment apparatus of claim 1 further comprising:
a flexible tube mounted between the main shaft and the mounting plug.

3. The deployment apparatus of claim 2, wherein the actuator rod comprises:
a first rod including the actuating pin;
a second rod including the actuator tip; and
a flexible joint connecting the first and second rods.

4. The deployment apparatus of claim 3, wherein the deployment apparatus further comprises:
a handle attached to the main shaft at an end opposite the flexible tube.

5. The deployment apparatus of claim 4, wherein the outer housing further comprises a grip on one end, the grip including a notch to engage the actuating pin.

6. The deployment apparatus of claim 5, wherein the deployment arms are connected to the mounting plug by pins, which allow the deployment arms to pivot on the mounting plug.

7. The deployment apparatus of claim 6, wherein the actuator tip comprises two notches.

8. The deployment apparatus of claim 7, wherein each deployment arm comprises a U-shaped tip for engaging a notch of the actuator tip.

9. The deployment apparatus of claim 6, wherein the actuator tip comprises a cap with two slots.

10. The deployment apparatus of claim 9, wherein each deployment arm comprises a pointed tip for engaging a slot in the actuator tip cap.

11. The deployment apparatus of claim 9, wherein each deployment arm comprises a rounded tip for engaging a slot in the actuator tip cap.

12. The deployment apparatus of claim 9, wherein each deployment arm comprises a square tip for engaging a slot in the actuator tip cap.

13. The deployment apparatus of 5, wherein the mounting plug is keyed to the actuator rod to prevent rotation of the actuator rod about a long axis.

14. A surgical mesh deployment system comprising:
a surgical mesh for insertion into a patient;
a surgical mesh deployment apparatus, the apparatus comprising:
a hollow main shaft having a slot on a proximal end;
an actuator rod positioned in the main shaft, the actuator rod having an actuating pin on a proximal end engaged through the slot in the main shaft, and an actuator tip at a distal end;
a mounting plug connected to an end of the main shaft, the mounting plug having a hole for the actuator rod;
two deployment arms connected to the mounting plug, wherein each deployment arm has a tip configured to engage the actuator tip; and
a hollow outer housing mounted over the main shaft, the outer housing having an interior diameter larger than an exterior diameter of the main shaft and mounting plug, such that the outer housing can slide along the main shaft and over the mounting plug and deployment arms;
wherein the surgical mesh is mountable on the deployment arms for deployment in a patient.

15. The deployment system of claim 14, wherein the deployment apparatus further comprises:
a flexible tube mounted between the main shaft and the mounting plug.

16. The deployment system of claim 15, wherein the actuator rod comprises:
a first rod including the actuating pin;
a second rod including the actuator tip; and
a flexible joint connecting the first and second rods.

17. The deployment system of claim 16, wherein the deployment apparatus further comprises:
a handle attached to the main shaft at an end opposite the flexible tube.

18. The deployment system of claim 17, wherein the outer housing further comprises a grip on one end, the grip including a notch to engage the actuating pin.

19. The deployment system of claim 18, wherein the deployment arms are connected to the mounting plug by pins, which allow the deployment arms to pivot on the mounting plug.

20. The deployment system of claim 14, wherein the surgical mesh is formed as an oval and comprises a plurality of guide loops.

21. The deployment system of claim 20, wherein the surgical mesh further comprises a pocket to receive the actuator tip.

22. The deployment system of claim 14, wherein the surgical mesh comprises two parallel oval sheets attached along the edges thereof with an opening on one end.

23. The deployment system of claim 22, wherein the surgical mesh further comprises at least one center guide loop and a pocket to receive the actuator tip.

24. The deployment system of claim 19, wherein the actuator tip comprises two notches.

25. The deployment system of claim 24 wherein each deployment arm comprises a U-shaped tip for engaging a notch of the actuator tip.

26. The deployment apparatus of claim 19, wherein the actuator tip comprises a cap with two slots.

27. The deployment apparatus of claim 26, wherein each deployment arm comprises a pointed tip for engaging a slot in the actuator tip cap.

28. The deployment apparatus of claim 26, wherein each deployment arm comprises a rounded tip for engaging a slot in the actuator tip cap.

29. The deployment apparatus of claim 26, wherein each deployment arm comprises a square tip for engaging a slot in the actuator tip cap.

30. The deployment system of claim 18, wherein the mounting plug is keyed to the actuator rod to prevent rotation of the actuator rod about a long axis.

31. A method of deploying a surgical mesh, the method comprising:
mounting a surgical mesh on two deployment arms of a deployment apparatus;
engaging the two deployment arm tips with an actuator tip of an actuator rod, the deployment arms attached to a fixed mounting plug at a first end, and releasably engaged with the actuator tip of the actuator rod at a second end;
sliding an outer housing over the surgical mesh and deployment arms to cover the mesh;
inserting the outer housing into a patient;
sliding a handle of the deployment apparatus forward to expose the surgical mesh and causing an actuating pin to engage a notch in a grip;
further sliding the handle forward causing a main shaft to slide over the actuator rod, which causes the deployment arms to expand outward to unfurl the surgical mesh,
pulling the handle back to release the deployment arm tips from the actuator rod tip, the actuating pin being engaged in the notch in the grip; and
removing the deployment apparatus from the patient.
